(19) United States
(12) Patent Application Publication
(10) Pub. No.: US 2009/0022270 A1
(43) Pub. Date: Jan. 22, 2009

(54) X-Ray Image Sensor and X-Ray Imaging Apparatus Using the Same

(75) Inventors: Takahiro Yoshimura, Kyoto (JP); Makoto Honjo, Kyoto (JP)

Correspondence Address: Quinn Emanuel Urquhart Oliver & Hedges LLP;Koda/Androlia, 865 S. Figueroa Street, 10th Floor, Los Angeles, CA, 90017, US

(73) Assignee: J. Morita Manufacturing Corporation

(21) Appl. No.: 11/918,090

(22) PCT Filed: Apr. 11, 2006
(86) PCT No.: PCT/JP2006/307676

§ 371 Date: Dec. 17, 2007

(30) Foreign Application Priority Data
Apr. 11, 2005 (JP) 2005-113834

(51) Int. Cl. A61B 6/08; A61B 6/14

(52) U.S. Cl. 378/19; 378/38

(57) ABSTRACT

An X-ray image sensor (1) for use in a medical X-ray imaging apparatus (10) has a mounting portion (12) for the X-ray image sensor (1) and a rotary means (13a) holding the X-ray image sensor (1) mounted on the mounting portion (12) and an X-ray generator (11) so as to interpose an object (H) to be examined therebetween. The X-ray image sensor (1) is so constructed as to be mounted on the mounting portion (12) and comprises an X-ray slit beam imaging plane (1S), (1P) which is vertically long and has small width and a CT imaging plane (1T) combined with said X-ray slit beam imaging plane (1S), (1P) and having larger width than the X-ray slit beam imaging plane.

### TECHNICAL FIELD

The present invention relates to an X-ray image sensor for use in an X-ray imaging of an object to be examined such as a dental arch, a head, and extremities of a human body and to an X-ray imaging apparatus using the sensor.

### BACKGROUND ART

As for the X-ray image sensor for use in the X-ray imaging apparatus, a vertically long sensor having a small width and an enough vertical length for including a head for an X-ray slit beam is required for a cephalometric radiography to obtain the transmission image of a head, a shorter sensor than the sensor for cephalometric radiography is required for a panoramic radiography to obtain a panoramic image of a dental arch, and a little wider sensor is required for a CT radiography to obtain a sectional image of a tooth.

However, for executing plural kinds of the above radiographies in such case of dental diagnosis, plural individual apparatuses have been prepared depending on the kinds of radiographies or otherwise an X-ray image sensor has been replaced depending on the radiography purposes.

On the other hand, a radiography method has been disclosed wherein a large-sized X-ray image sensor is mounted in advance, one X-ray image sensor is partially masked depending on radiography purposes, and the masked portion is varied following the kinds of radiographies (Patent Document 1). The patent document 2 discloses an example of prior X-ray imaging apparatus.
Patent Document 1: JP-A-10-225455
Patent Document 2: JP-A-07-275240

### DISCLOSURE OF THE INVENTION

### Problems to be solved in the Invention

Such a kind of large-sized image sensor is effective for a user to operate, however, it requires for a manufacturer to prepare a large-sized X-ray image sensor, thereby reducing the production yield and causing a high cost.

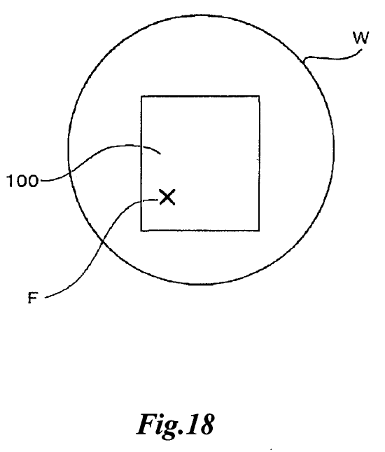

In case of a large X-ray image sensor 100, as shown in FIG. 18, the image sensor cannot be efficiently cut out of a semiconductor wafer and if there is a damage F only on a part of the sensor 100, the sensor becomes defective, so that the production yield is reduced to result in high production costs.

The present invention is proposed considering the above and its first object is to provide an X-ray image sensor which can improve the production yield ratio for a manufacturer and can give convenience for a user.

In these days, an X-ray imaging apparatus capable of any one of a cephalometric radiography, a panoramic tomography, a linear tomography, and a CT radiography has been developed. The second object of the present invention is to use one X-ray image sensor for various radiography purposes by means of a simple method.

### Means to Solve the Problem

In order to achieve the above-mentioned objects, according to claim 1, an X-ray image sensor for use in a medical X-ray imaging apparatus comprising a rotary means which has a mounting portion for the X-ray image sensor and holds the X-ray image sensor mounted on the mounting portion and an X-ray generator so as to interpose an object to be examined between the X-ray image sensor and the X-ray generator is characterized in that the X-ray image sensor is so constructed as to be mounted on the mounting portion, and comprises an X-ray slit beam imaging plane which is vertically long and has small width, and a CT imaging plane combined with the X-ray slit beam and having larger width than the X-ray slit beam imaging plane.

According to claim 2, the CT imaging plane is combined with the X-ray slit beam imaging plane in a manner that the CT imaging plane intersects with the X-ray slit beam imaging plane.

According to claim 3, the X-ray slit beam imaging plane has a longitudinal dimension required for a cephalometric radiography, and is so constructed as to be masked in a part of the X-ray slit beam imaging plane with a shielding member when a panoramic radiography or a liner tomography is executed.

According to claim 4, both of the CT imaging plane and the X-ray slit beam imaging plane are composed of any one selected from a MOS sensor, a CMOS sensor, a TFT sensor, an X-ray solid-state image sensing device, and a CCD sensor.

According to claim 5, an X-ray imaging apparatus comprises a rotary means holding an X-ray generator for interposing an object to be examined together with the imaging sensor as set forth in any one of claims 1-4 between them, and an orbit control means for moving the rotary means along any one of different kinds of orbits prepared in advance in order to produce an X-ray image of the object for different diagnosis purposes.

According to claim 6, only the X-ray slit beam imaging plane of the X-ray imaging sensor is used and opened as an effective imaging plane depending on the width of an X-ray slit beam corresponding to the kinds of the radiographies when either a linear tomography, a panoramic tomography, or a cephalometric radiography is executed by radiating X-ray slit beam on the object to be examined, while only the CT imaging plane of the X-ray imaging sensor is used and opened when X-ray CT scan is executed, and the orbit control means moves the rotary means relative to the object along the orbit respectively for performing at least two types of tomography selected from a linear tomography, a panoramic tomography, a cephalometric radiography, and an X-ray CT scan.

According to claim 7, the X-ray imaging apparatus of claim 5 or 6 has an imaging means for producing a scout view image, and wherein the resolution is reduced in case of producing a scout view image.

According to claim 8, the X-ray imaging apparatus of claim 5 or 6 has an imaging means for executing any one of a linear tomography, a panoramic tomography, a cephalometric radiography, and an X-ray CT scan, and wherein the resolution is reduced in case of producing an image by way of any one of the linear tomography, the panoramic tomography, the cephalometric radiography, and the X-ray CT scan.

### Effect of the Invention

According to the X-ray image sensor of the present invention, a CT imaging plane is connected with an X-ray slit beam imaging plane which is vertically long and has small width, the CT imaging plane having longer width than the X-ray slit beam imaging plane. Therefore, the image sensor can be used for an X-ray slit beam imaging such as a cephalometric radiography and a panoramic radiography and also for a CT imaging. Even when plural kinds of radiographies are required in such a dental diagnosis, only one kind of image sensor is prepared to be selectively used for plural radiographies by a simple method.

As for a manufacturer, it is only required to produce an X-ray slit beam imaging plane and a CT imaging plane, which have different dimensions, individually and to connect the planes, so that more sensors can be produced from one semiconductor wafer, thereby increasing the production yield.

Specifically, if an image sensor is constructed by connecting a segment smaller than each imaging plane, the production yield shall be further improved.

Further according to the present invention, the CT imaging plane is connected so as to intersect with the X-ray slit beam imaging plane, thereby facilitating production and usage.

Still further according to the present invention, the X-ray slit beam imaging plane has a longitudinal dimension required for a cephalometric radiography, and a part thereof is designed to be masked with a shielding member in case of a panoramic radiography and a liner tomography, so that one kind of image sensor can be appropriately used depending on a desired radiography.

Still further according to the X-ray imaging apparatus of the present invention, because the above-mentioned the X-ray image sensor is provided, an X-ray image of an object to be examined for different diagnosis purposes can be efficiently obtained. Specifically, the apparatus facilitates the switch operation of radiography, because the apparatus has a rotary means holding an X-ray generator and because the X-ray imaging apparatus so as to interpose an object to be examined, and an orbit control means for moving the rotary means following any one of different kinds of orbits prepared in advance in order to obtain an X-ray image of an object to be examined for different diagnosis purposes.

Still further according to the present invention, a binning process can be adopted for reducing the resolution. In the process, the radiography charge which is the output of image sensor is superimposed, so that the X-ray dosage irradiated from the X-ray generator can be reduced or the rotary speed of the rotary means can be increased anticipating the increasing amount of radiography charge after the process, thereby reducing the X-ray exposure dosage.

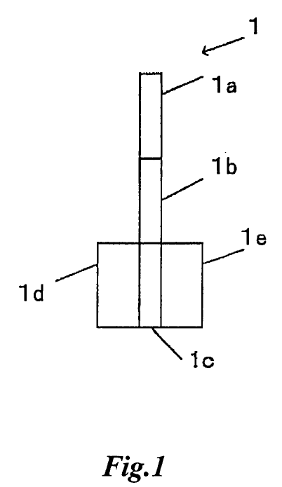

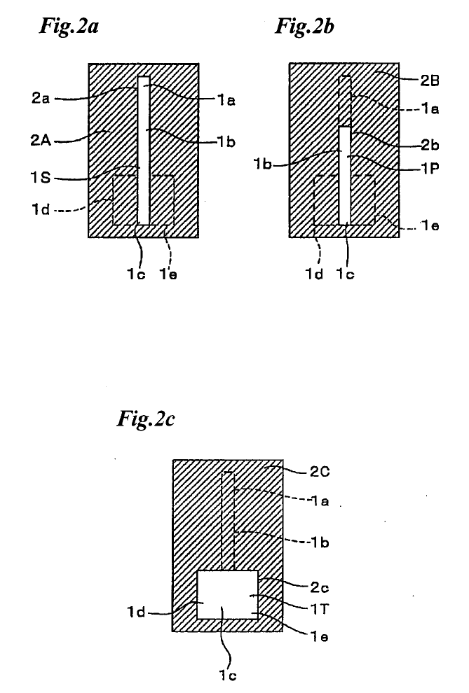

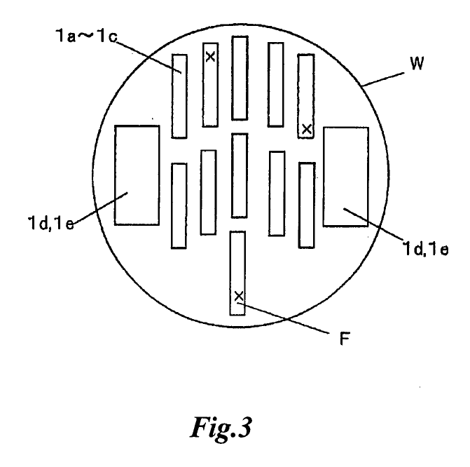

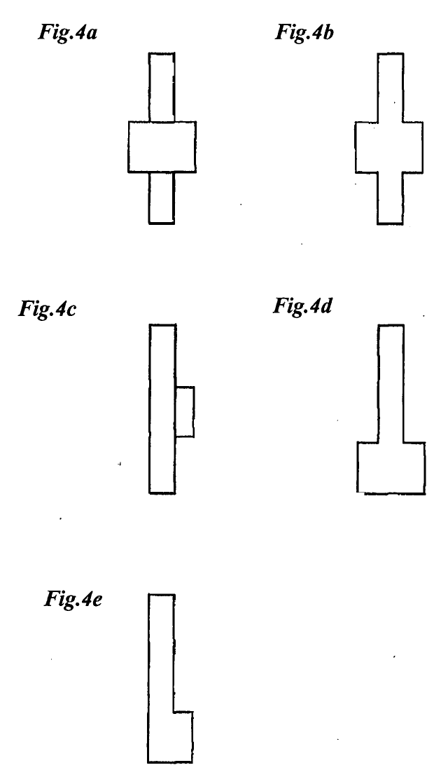

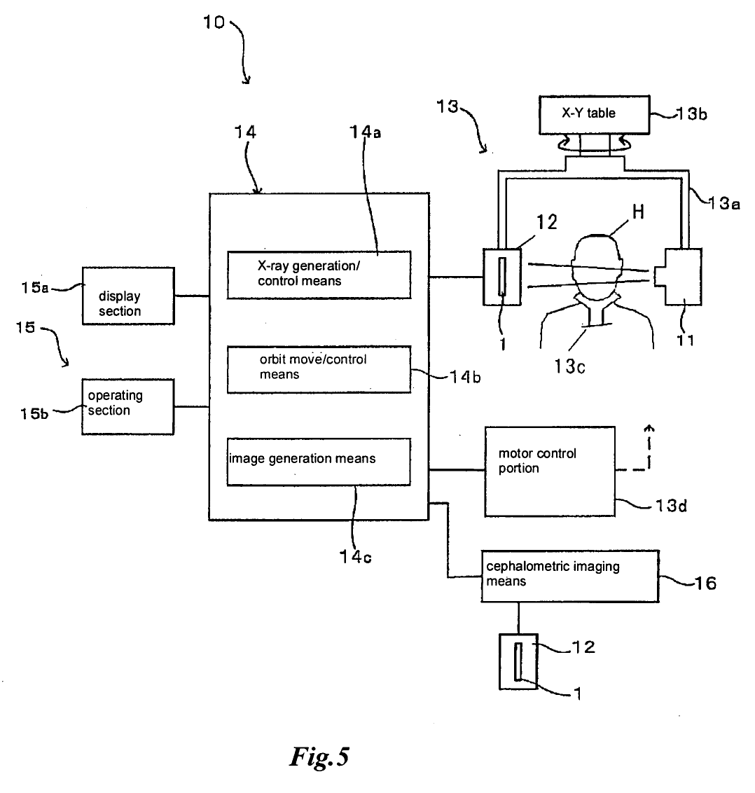

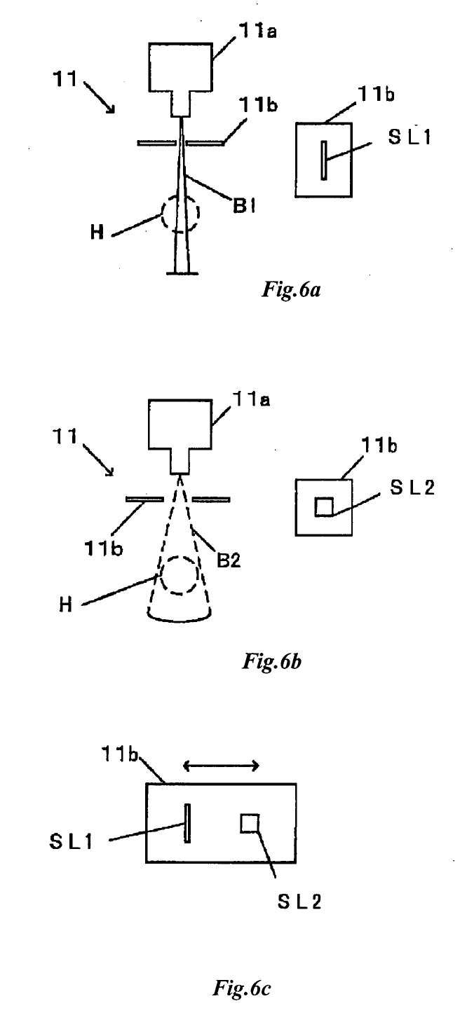

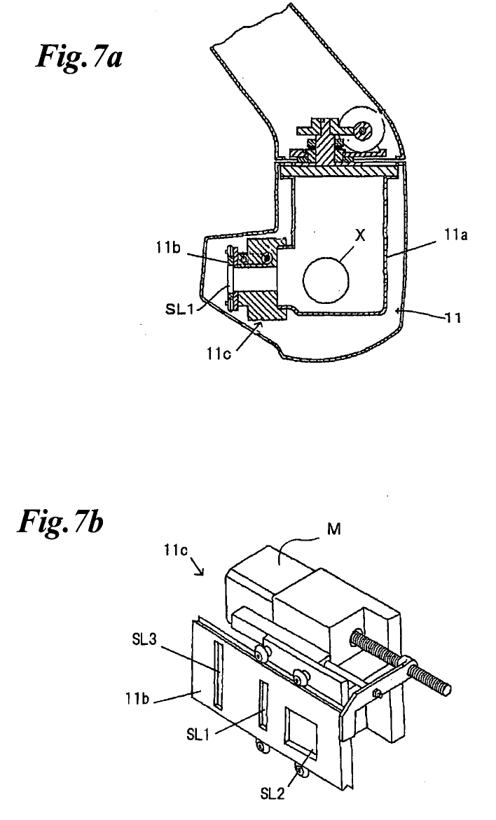

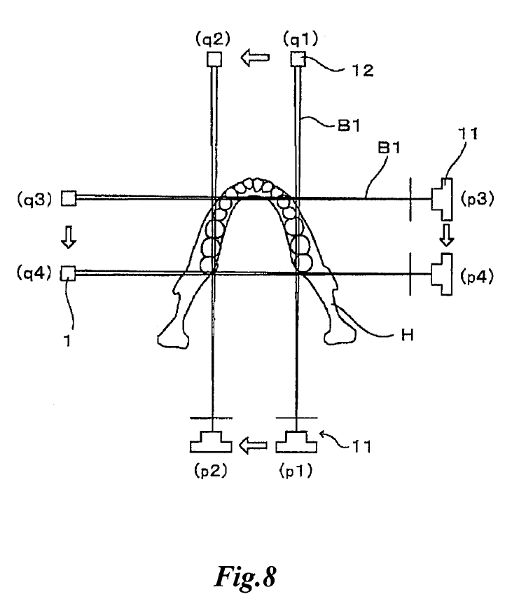

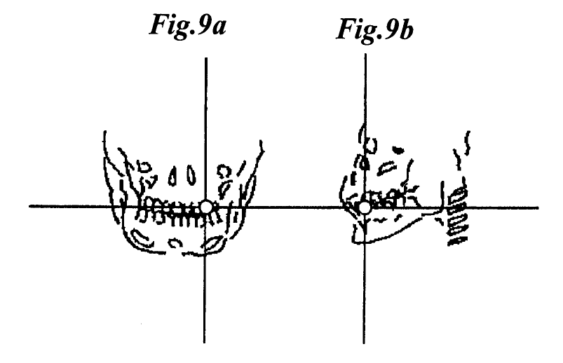

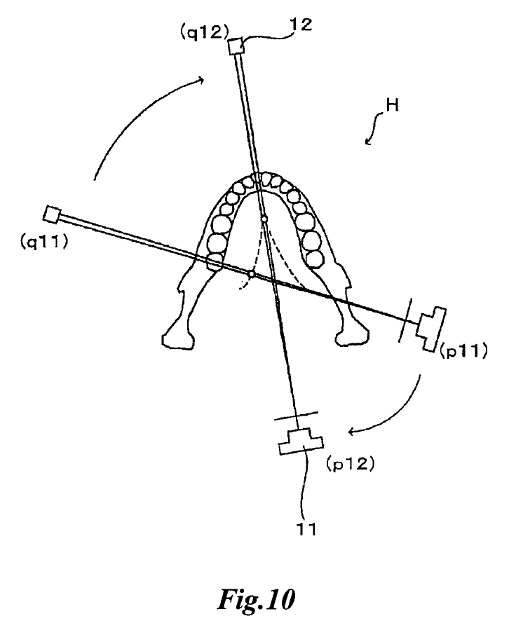

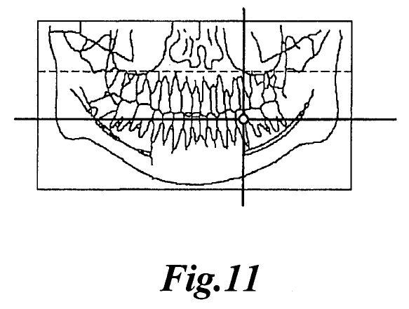

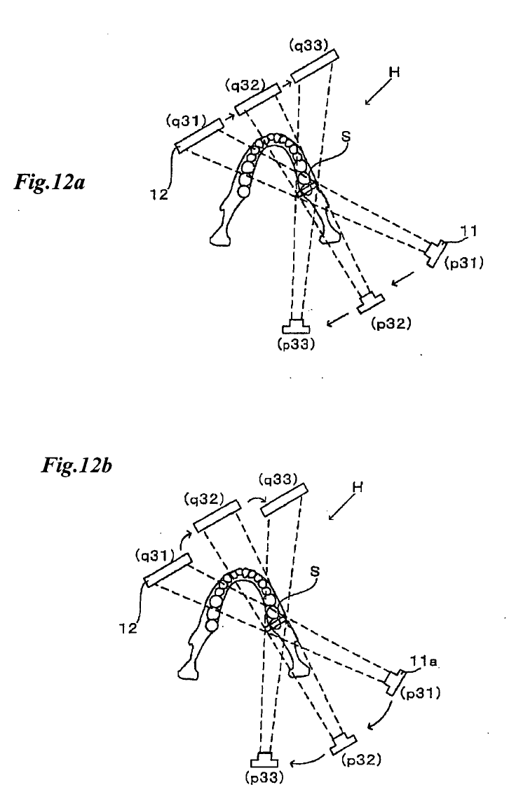

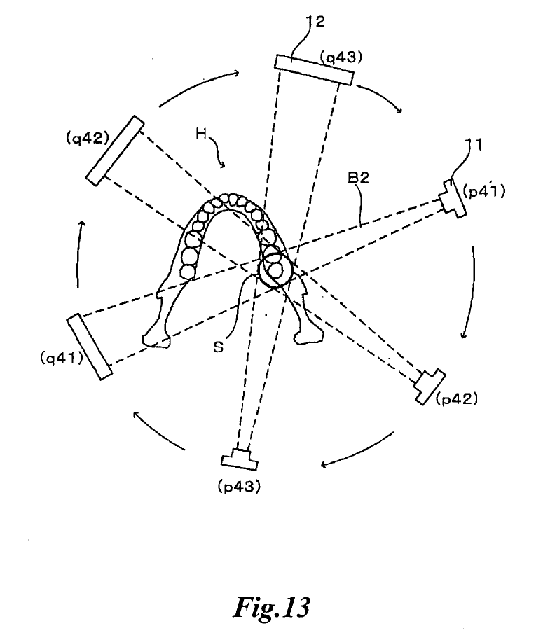

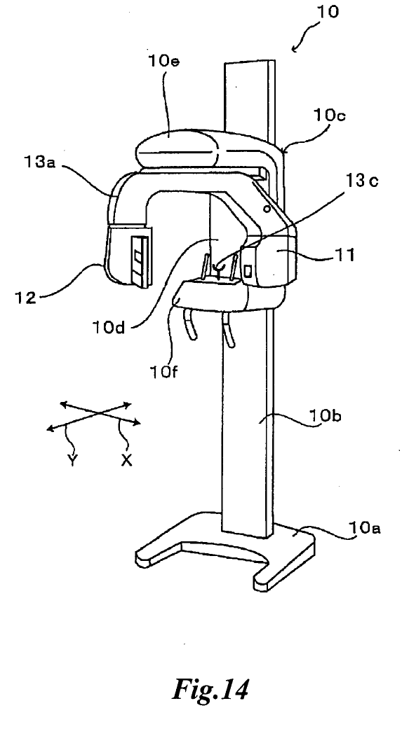

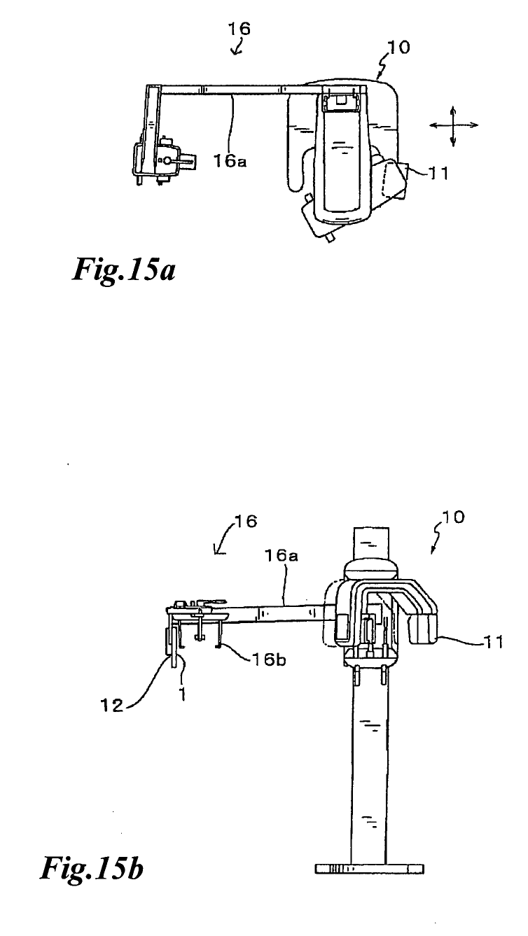

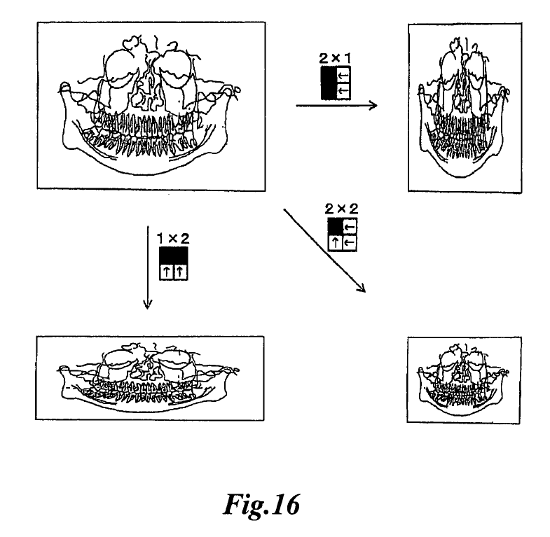

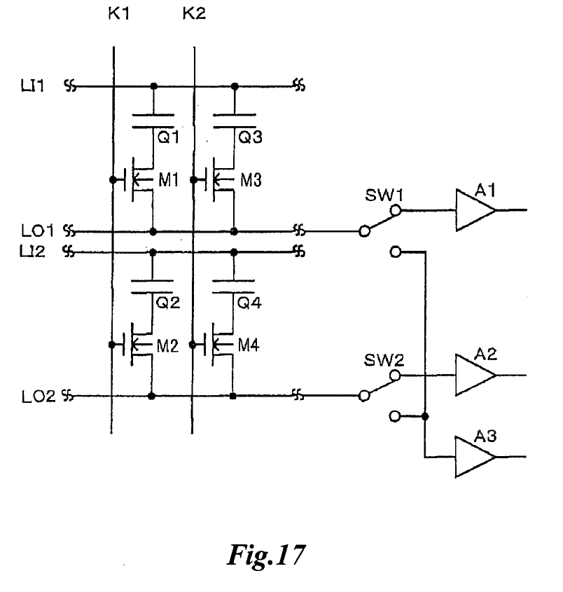

### BRIEF DESCRIPTION OF DRAWINGS

[FIG. 1] shows a planar shape of an X-ray image sensor which is one example of the present invention.

[FIG. 2] is an explanatory view how the image sensor shown in FIG. 1 is used, FIG. 2a shows an imaging plane for a cephalometric radiography, FIG. 2b shows an imaging plane for a panoramic radiography, and FIG. 2c shows an imaging plane for a CT radiography

[FIG. 3] shows how an image sensor of the present invention is produced from a semiconductor wafer.

[FIG. 4] shows an other shape of an X-ray image sensor of the present invention.

[FIG. 5] shows a block diagram of an essential structure of an X-ray diagnosis imaging apparatus of the present invention.

[FIG. 6] is an explanatory view of an X-ray generation principle by means of an X-ray generator.

[FIG. 7] explains a specific structure of an X-ray generator, FIG. 7a is a vertical section and FIG. 7b is a perspective view.

[FIG. 8] is a plain view of an orbit for obtaining a liner scan image.

[FIG. 9] shows an example of a linear scan image of a human lower jaw, FIG. 9a shows a front view, and FIG. 9b shows a side view.

[FIG. 10] is a plain view of an orbit for obtaining a panoramic image.

[FIG. 11] shows an example of a panoramic image of a human dental arch.

[FIG. 12] is a plain view of an orbit for obtaining a liner tomographic image.

[FIG. 13] is a plain view of an orbit for obtaining a CT tomographic image.

[FIG. 14] shows an external view of one example of an X-ray diagnosis imaging apparatus of the present invention.

[FIG. 15] shows an X-ray imaging apparatus attached with a cephalometric imaging means, FIG. 15a is a plain view and FIG. 15b is a side view.

[FIG. 16] is a view for explaining a binning process.

[FIG. 17] is a simplified circuit diagram of a CMOS sensor.

[FIG. 18] shows how a large image sensor is produced from a semiconductor wafer.

### REFERENCE NUMBER

1 X-ray image sensor
1S cephalometric imaging plane
1P panoramic imaging plane
1T CT imaging plane
2A, 2B, 2C shielding member
10 X-ray imaging apparatus
11 X-ray generator
12 sensor mounting portion
13 moving means
13a rotary means
14 X-ray imaging control means
14b orbit control means
16 cephalometric imaging means
H object to be examined

### BEST MODE FOR CARRYING OUT THE INVENTION

The embodiments of the present invention are explained hereinafter referring to the attached drawings.

### Embodiment 1

FIG. 1 shows a planar shape of an X-ray image sensor which is one example of the present invention.

The X-ray image sensor 1 is a MOS type semiconductor sensor and is used as an imaging plane of an X-ray imaging apparatus capable of a cephalometric radiography, a panoramic radiography, a linear tomography, and a CT radiography. The sensor 1 may be a CMOS sensor, a TFT sensor, an X-ray solid-state image sensing device and a CCD sensor, other than a MOS sensor.

The structure of the X-ray imaging apparatus and the principal of each radiography are explained later.

The X-ray image sensor 1 is constructed such that five segments 1a-1e are connected to form a reverse letter T as shown in the figure.

In the figure, assuming a dental X-ray imaging, small width segments 1a-1c have a width of about 6 mm, 1d and 1e have a width of about 35 mm, there length is about 75 mm, however, the present invention is not limited to such segments.

The segments 1a, 1b and 1c in the X-ray image sensor 1 are used as a slit scan beam imaging plane and the segments 1c, 1d, and 1e formed at a lower part are utilized as an imaging plane for a CT radiography using a broad scan beam.

Specifically in the figure, a conical beam having relatively thick beam flux and small width is assumed for the imaging plane for a CT radiography in order to reduce the exposed dose, however, it goes without saying that the present invention is not limited to such an imaging plane because the dimension of imaging plane is specified depending on the radiography purposes.

FIG. 2 is an explanatory view showing how the image sensor shown in FIG. 1 is used, FIG. 2a shows an imaging plane for a cephalometric radiography, FIG. 2b shows an imaging plane for a panoramic radiography, and FIG. 2c shows an imaging plane for a CT radiography. The figures also show each shielding member. The reference numerals 2A-2C are shielding member constructed so as to cover a part of the image sensor 1, the reference numerals 2a-2c are openings provided for each shielding members 2A-2C for exposing an imaging plane. The dotted lines show an unexposed portion of the X-ray image sensor.

In FIG. 2a, the segments 1a-1c are exposed by the opening 2a and other segments 1d and 1e are masked, thereby forming a cephalometric imaging plane 1S for a cephalometric radiography. In FIG. 2b, the segments 1b and 1c are exposed by the opening 2b and other segments 1a, 1d and 1e are covered, thereby forming a panoramic imaging plane 1P for a panoramic radiography. In FIG. 2c, the segments 1d, 1c and 1e are exposed by the opening 2c and other segments 1a and 1b are covered, thereby forming a CT imaging plane 1T for a CT radiography.

Although it is not shown in the figure, the imaging planes required for a linear tomography are exposed being masked by other shielding members in case of a linear tomography.

The shielding members 2A-2C are appropriately replaced as mentioned above, one X-ray image sensor 1 can be used for several kinds of radiographies. Therefore, it is not required to replace and use several kinds of X-ray image sensors, thereby facilitating management. The X-ray image sensor 1 is designed to have minimum size and shape required for a cephalometric radiography, a panoramic tomography, a CT radiography, and a linear tomography, thereby minimizing the portion which is not used for radiography and avoiding waste.

As for a manufacturer, different sized X-ray slit beam imaging planes (cephalometric imaging plane 1S and a panoramic imaging plane 1P) and a CT imaging plane 1T can be individually manufactured and connected, so that he can produce many sensors from a sheet of semiconductor wafer W (see FIG. 3).

Further, the X-ray image sensor 1 can be formed with a combination of small segments 1a-1e as in this embodiment, so that when the segments 1a-1e are formed by cutting out of the semiconductor wafer W as shown in FIG. 3, the wafer W can be effectively utilized. Still further, if there is a damage F on any one of segments on the semiconductor wafer W and such segment becomes defective, other segments are not affected, thereby improving process yield.

The X-ray image sensor 1 is not limited to the above-mentioned shape and structure, and it may have other shape and structure. At least it is required to have a wide shaped portion for a broad scan beam and a long portion for a slit scan beam. Also it is required that a broad CT imaging plane 1T and a long X-ray slit beam imaging plane (a cephalometric imaging plane 1S and a panoramic imaging plane 1P) are connected. Plural segments may be connected as shown in the above embodiment or the whole may be integrally formed.

FIG. 4 shows other shape of an X-ray image sensor 1. In FIG. 4a, plural segments are connected in the form of cross, in FIG. 4b, they are integrally formed like a cross, in FIG. 4c, they are connected like a Japanese Katakana character “ [symbol] ”, in FIG. 4d, they are integrally formed like a reverse letter T, and in FIG. 4e, they are integrally formed like a letter L.

The structure of an X-ray imaging apparatus using the above-mentioned X-ray image sensor 1.

FIG. 5 shows a block diagram of an essential structure of an X-ray imaging apparatus of the present invention. FIG. 6 is an explanatory view showing the structure of an X-ray generator 11 used for the X-ray imaging apparatus 10.

As shown in the figure, the X-ray imaging apparatus 10 comprises a moving means 13 which holds an X-ray generator 11 and a sensor mounting means 12 for the X-ray image means 1 so as to face each other interposing an object to be examined H such as a human head, an X-ray imaging control means 14 for controlling the X-ray generator 11, the sensor mounting portion 12 and the moving means 13, a radiography selection means 15, and a cephalometric imaging means 16 having other X-ray image sensor 1 (and the sensor mounting portion 12 mounting the sensor 1) for a cephalometric radiography.

The X-ray generator 11 comprises an X-ray source 11a for generating X-rays by an X-ray tube current or an X-ray tube voltage controlled by the X-ray imaging control means 14, a collimator (not shown) for taking out the X-rays emitted from the X-ray source 11a, and a first slit plate 11b for regulating the irradiation area of X-rays.

The first slit plate 11b shown in FIG. 6a is designed such that a vertically-long narrow slit SL1 (horizontal to vertical ratio is about from 1:20 to 1:100) is formed on an X-ray shielding plate, by which the X-rays generated by the X-ray source 11a are regulated its irradiation area by the narrow slit SL1 and irradiated to an object to be examined H as an X-ray slit beam B1 which is vertically long and narrow in width. On the other hand, the first slit plate 11b shown in FIG. 6b is designed such that a rectangular slit SL2 (horizontal to vertical ratio is about from 1:1 to 2:1) is formed on an X-ray shielding plate, and the X-rays generated by the X-ray source 11a are regulated its irradiation area by the rectangular slit SL2 and irradiated to an object to be examined H as an X-ray broad beam B2 which is spreading at a fixed range.

According to the X-ray generator 11 adopting the first slit plate 11b as shown in FIG. 6a and FIG. 6b, the X-ray slit beam B1 or X-ray broad beam B2 is selectively switched to be generated by selecting either one of two first slit beams 11b shown in FIG. 6a and FIG. 6b by means of the X-ray imaging control means 14.

The first slit plate 11b shown in FIG. 6c is designed such that the above-mentioned narrow slit SL1 and the above-mentioned rectangular slit SL2 are both formed on one X-ray shielding plate. By means of the X-ray generator 11 adopting such a first slit plate 11b, an actuator (not shown) is driven by the X-ray imaging control means 14 to make the first slit plate 11b provided in front of the X-ray source 11a slide from side to side, as the result, the X-ray narrow beam B1 or the X-ray broad beam B2 is selectively switched and generated.

The sensor mounting portion 12 is attached with the X-ray image sensor 1, a part of which is masked with the shielding member 2A-2C as mentioned referring to FIG. 2 corresponding to the X-ray narrow scan beam B1 and the X-ray broad beam B2 irradiated from the X-ray generator 11.

FIG. 7a and FIG. 7b Show a vertical section and a perspective view respectively for explaining a specific structure of the X-ray generator 11. As shown in the figure, the X-ray source 11a including an X-ray tube bulb X is incorporated in a housing including the X-ray generator 11 as shown in the figure. Provided in front of the X-ray source 11a are the first slit plate 11b comprised of an X-ray shielding plate formed with plural first slits and a slit module 11c including an adjustment mechanism for changing the shape of the first slit. The first slit plate 11b in this embodiment is provided with a narrow slit SL1 for a panoramic radiography, a rectangular slit SL2 for a CT radiography (CT), and a long slit SL3 for a cephalometric radiography and when a cassette is changed, the slit module 11c sets a first slit corresponding to the changed cassette by sliding the first slit 11b by means of a driving motor M.

The moving means 13 comprises a rotary means 13a having the X-ray generator 11 and a mounting portion 12, a shaft moving platform 13b having an X-Y table for horizontally moving the shaft of the rotary means 13a while keeping vertical suspending position in a rotatable manner, and a positioning means 13c for positioning the object to be examined H. Rotation of the rotary means 13a and the horizontal movement of the rotary shaft of the rotary means 13a are driven by a respective stepping motor controlled by the X-ray imaging control means 14. Further, the positioning means 13c may be moved up and down by the similar stepping motor. The rotary means 13a is not limited to a rotary arm suspending the X-ray generator 11 and the sensor mounting portion 12 face to face while interposing the object to be examined H as shown in the figure.

The X-ray imaging control means 14 is connected with a motor control portion 13d having a stepping motor for driving the moving means 13, a display 15a for showing the information such as X-ray images on a monitor television, and an operating section 15b for receiving the operations of a key board or a mouse. Further the X-ray imaging control means 14 is provided with an X-ray generation control means 14a which controls the X-ray tube current or the X-ray tube voltage of the X-ray generator 11 as a mechanical element and selectively switches and generates the X-ray slit beam B1 or the X-ray broad beam B2, an orbit control means 14b for moving the moving means 13 by controlling the motor control portion 13d and for moving the X-ray generator 11 and the sensor mounting portion 12 along the orbit depending on the kinds of radiographies, and an image production means 14c for producing a transmission image or a sectional image from the obtained X-ray image data.

The display 15a and the operating section 15b construct a radiography selection means 15 wherein a transmission image which has been obtained before an objective sectional image is shown as a broad transmission image of the object to be examined H, namely a scout view image, a sectional image or an diagnostic region to be obtained its sectional image inside of the object to be examined H is selected as an interested area “s”, and the kinds of radiography of the sectional image of the interested area “s” are selected.

Next, the basic operation such as obtaining a scout view image, selecting the kind of radiography, and obtaining a sectional image, of the X-ray imaging apparatus 10 is sequentially explained.

In case of obtaining a scout view image, it is characterized in that the object to be examined H is scanned by means of the X-ray slit beam B1 while synchronously moving the X-ray generator 11 and the sensor mounting portion 12 along a fixed orbit and its transmission image is obtained. As such a scout view image, a linear scan image and a panoramic image can be utilized and selection of radiography, namely which images are used, is set in advance by means of the radiography selection means 15.

In this radiography, the orbit control means 14b reads out the orbit data stored in an orbit memory (not shown) and controls the moving means 13 via the motor control portion 13d, thereby synchronously moving the X-ray generator 11 and the mounting portion 12 along a fixed orbit. The X-ray generation control means 14a makes the X-ray slit beam B1 irradiate from the X-ray generator 11 to scan the object to be examined H following the intensity data stored in an irradiation intensity memory (not shown), namely profile.

After finishing the radiography, the image generation means 14c arranges a series of transmitted data in time series to produce a scout view image.

In case of selecting the kinds of radiographies, a linear scan image or a panoramic image which is obtained as a scout view image is shown on the display 15a together with a cursor movable on the image. For example, if the cursor is moved to a specific sectional image or a diagnostic region by means of a mouse of the operation part 15b and the mouse is clicked, the clicked portion is established as an interested area “s”. Then the kinds of radiographies of the sectional image to be obtained on the interested area “s” by a predetermined key operation, the selected tomography is started. A linear sectional image, a CT image, a panoramic sectional images and so on can be selected as a sectional image of the obtained transmission image.

In case of obtaining a sectional image, an X-ray broad beam B2 is irradiated from the X-ray generator 11 while synchronously moving the X-ray generator 11 and the sensor mounting portion 12 along a fixed orbit, a transmission image of the object to be examined H is obtained at plural times as a frame with a fixed expanse by the CT imaging plane 1T exposed by the opening 2c of the sensor mounting portion 12, and plural transmission images are obtained depending on the position of the orbit. Thereafter, the sectional image of the interested area “s” is obtained by an image processing such as composition or arithmetic processing.

In this radiography, the orbit control means 14b reads out the orbit data stored in the orbit memory and controls the moving means 13 via the motor control portion 13d, thereby synchronously moving the X-ray generator 11 and the sensor mounting portion 12 along a fixed orbit. The X-ray generation control means 14a irradiates an X-ray broad beam B2 from the X-ray generator 11 on the interested area “s” of the object to be examined H along the intensity data registered in the irradiation intensity memory, namely profile at a fixed position on the orbit, and simultaneously the orbit control means 14b makes the X-ray image sensor measure the X-ray transmitted through the interested area “s” to send the transmission image to an image layer production means 14d each time of measuring. Finishing this radiography, the image production means 14c executes a fixed process for the plural transmission images which have been sent, thereby producing a sectional image of the interested area “s”.

How the scout view image is obtained is explained referring to the drawings using an example of the orbit and the obtained transmission images in case of obtaining a linear scan image or a panoramic image.

FIG. 8 is a plain view explaining an orbit for obtaining a liner scan image. FIG. 9 shows an example of the linear scan image. In FIG. 8 a lower jaw is imaged as the object to be examined H and in FIG. 9 a crosshair cursor for specifying the interested area “s” is shown together with the linear scan image.

In this radiography, the X-ray generator 11 and the sensor mounting portion 12 with the X-ray image sensor 1 are faced each other interposing the object to be examined H and synchronously moved in parallel while irradiating an X-ray slit beam B1 at an equal angle and measuring the X-rays transmitted through the object to be examined H.

More specifically, the orbit control means 14b moves the X-ray generator 11, which is designed to irradiate an X-ray slit beam B1, along the orbit from a position (p1) to a position (p2) by controlling the moving means 13 and synchronously moves the sensor mounting portion 12 with the X-ray image sensor 1 along the orbit from a position (q1) to a position (q2). In this case, the X-ray slit beam B1 transmits through the object to be examined H in a vertical direction, so that a front linear scan image of the object to be examined H as shown in FIG. 9a can be obtained.

Similarly, the orbit control means 14b moves the X-ray generator 11, which is designed to irradiate an X-ray slit beam B1, along the orbit from a position (p3) to a position (p4) and synchronously moves the sensor mounting portion 12 with the X-ray image sensor along the orbit from a position (q3) to a position (q4). In this case, the X-ray slit beam B1 transmits through the object to be examined H in a crosswise direction, so that a side linear scan image of the object to be examined H as shown in FIG. 9b can be obtained. Thus obtained front and side linear scan images are shown on the display 15a at the same time to be utilized for setting an interested area “s”.

FIG. 10 is a plain view of an orbit by which the X-ray generator 11 and the sensor mounting portion 12 are simultaneously moved for obtaining a panoramic image. FIG. 11 shows an example of an obtained panoramic image.

In this radiography, the X-ray slit beam B1 is irradiated along the orbit which is directed to be incident in substantially vertical to each part of the dental arch being the object to be examined H, thereby scanning and obtaining plural transmission images. Thus obtained transmission images are combined and a panoramic image is produced.

More specifically, the orbit control means 14b moves the X-ray generator 11 which is designed to irradiate an X-ray slit beam B1 along the orbit from a position (p11) to a position (p12) by controlling the moving means 13 and synchronously moves the sensor mounting portion 12 with the X-ray image sensor 1 along the orbit from a position (q11) to a position (q12). In this scan imaging, a panoramic image of the object to be examined H as shown in FIG. 11 can be obtained. The dotted lines in FIG. 10 shows an orbit of a rotary shaft of the rotary means 13a. A method for producing a scout view image is explained taking a linear scan image and a panoramic image for example as mentioned above, however, a scout view image is not limited to a linear scan image or a panoramic image and it may be a cephalometric image by means of a cephalometric imaging means as mentioned later.

Next, radiography of a sectional image is explained taking a orbit in case of obtaining a linear sectional image and a CT image for example.

FIG. 12a and FIG. 12b show a plane view showing two kinds of orbits for synchronously moving the X-ray generator 11 and the sensor mounting portion 12 for obtaining a linear sectional image. A sectional plane is set for the object to be examined H as an interested area “s”.

In a linear tomography, an X-ray slit beam B1 is irradiated by changing the irradiation angle from the X-ray generator 11 to the sectional plane being the interested area “s” to produce plural transmission images of the object to be examined H and a sectional image can be obtained by overlapping the produced transmission images so as to emphasize a fixed sectional plane from the transmission images.

Namely in the example of FIG. 12a, the orbit control means 14b moves the X-ray generator 11, which is designed to irradiate an X-ray broad beam B2, along the orbit from a position (p31) to a position (p33) by controlling the moving means 13 and synchronously 1 moves the sensor mounting portion 12 with the X-ray image sensor along the orbit from a position (q31) to a position (q33). Thus obtained transmission images by the radiography along the orbit are overlapped each other according to a fixed positional relation, then a linear sectional image can be synthesized. Synthesis of linear sectional images is the same as the prior art, therefore its explanation is omitted here.

FIG. 12b shows an example of an orbit different from FIG. 12a. In FIG. 12a the X-ray generator 11 and the X-ray image sensor 1 move rectilinear in a reverse direction each other, however in FIG. 12b, the X-ray generator 11 and the X-ray image sensor 1 move describing an arc in a reverse direction each other.

FIG. 13 shows a plane view showing an orbit for synchronously moving the X-ray generator 11 and the sensor mounting portion 12 for obtaining a CT image. A cylindrical diagnostic region is set for the object to be examined H as an interested area “s”.

In this case, the orbit control means 14b moves the X-ray generator 11, which is designed to irradiate an X-ray broad beam B2, along the orbit from a position (p41) to a position (p43) by controlling the moving means 13 and synchronously moves the sensor mounting portion 12 with the X-ray image sensor 1 along the orbit from a position (q41) to a position (q43). Thus obtained transmission images by the radiography along the orbit are back projected according to a well-known method, then a CT image of the interested area “s” can be combined. An orbit forming at least more than 180 degrees is required for obtaining a CT image.

FIG. 14 shows an entire perspective view of other example of an X-ray imaging apparatus.

The X-ray imaging apparatus 10 has a base board 10a fixed on the floor in the dental clinic, a support pillar 10b vertically established on the base board, and an elevation unit 10c movable up and down along the pillar 10b by a motor control portion 13d (see FIG. 5). The elevation unit 10c comprises a main frame 10d, an upper frame 10e projected forward from the upper part of the main frame 10d and a lower frame 10f projected forward from the lower part of the main frame 10d. The upper frame supports a rotary means 13a comprised of a rotary arm and the lower frame 10f has a positioning means 13c constituted as a chinrest for holding the object to be examined H, for example a human head.

The chinrest is movable up and down or inclinable so as to be positioned according the size of a patient. Such a movable structure can adjust the inclination of an irradiated line relative to a horizontal plane per a radiography region such as an upper jaw or a lower jaw and can adjust positioning to include in an irradiation field the apart regions such as an articulation of jaw positioned upper part and the tip of a lower jaw positioned lower part.

Here the structure of the elevation unit 10c and the lower frame 10f is explained.

The elevation unit 10c moves up and down relative to the support pillar 10b according to the size of a patient. The elevation unit 10c and the lower frame 10f are integrally formed. Therefore, the X-ray generator 11 and the X-ray image sensor 1 can be moved up and down together with the lower frame 10f and the positioning means 13c.

However, the lower frame 10f and the elevation unit 10c which accompanies elevation of the X-ray generator 11 and the X-ray image sensor 1 are separately constructed and either one of them may be moved independently relative to the pillar 10b. Otherwise, it may be constructed such that the X-ray generator 11 moves relative to the lower frame 10f or the positioning means 13c. JP-A-7-275240 which has been filed by the applicant of the present invention discloses an embodiment in which the above-mentioned lower frame 10f and the elevation unit 10c are separately constructed or an embodiment in which the X-ray generator 11 moves relative to the lower frame 10f and the positioning means 13c.

In JP-A-7-275240, the above mentioned lower frame 10f is referred as “a patient frame” and the elevation unit 10c is referred as “an elevation body”. There objects are to extend a radiography area, to adjust the inclination of an irradiation line relative to a horizontal plane per a radiography region such as an upper jaw and a lower jaw, and to position apart regions such as an articulation of jaw positioned upper part and the tip of a lower jaw positioned lower part.

The structure in which the positioning means 13c is movable up and down or inclinable, the structure in which the above-mentioned lower frame 10f and the elevation unit 10c are provided separately, or the structure in which the X-ray generator 11 is movable relative to the lower frame 10f and the positioning means 13c may be combined so as to execute more minute adjustment.

FIG. 15a and FIG. 15b show a plain view and a side view of an X-ray imaging apparatus 10 further attached with a cephalometric imaging means 16.

The X-ray imaging apparatus 10 is constructed such that a cephalometric imaging means 16 is further provided with the structure shown in FIG. 14. The cephalometric imaging means 16 has a holding arm 16a, a head fixing device 16b and a sensor mounting portion 12 with the X-ray image sensor 1.

According to the cephalometric radiography with the cephalometric imaging means 16, a head being an object to be examined H is fixed with the head fixing means 16b and the X-ray image sensor 1 is moved while keeping the X-ray generator 11 directing into the X-ray image sensor 1 of the cephalometric imaging means 16, thereby executing scan.

The wide transmission image of an object to be examined H, namely a scout view, according to the present invention has an object to set an interested area “s” being an object of radiography of a sectional image on the object to be examined H. For this purpose, it only requires to select a specified region from the entire image, and it is not always necessary to have a high resolution image. Therefore, it is preferable to select an appropriate resolution when necessary in obtaining a scout view. Such a structure is worth to reducing the exposure amount.

In order to be able to select the resolution of a scout view, a binning process, which is known as a prior art, can be introduced. The binning process is easily executed such that basically, a CCD sensor is used as an image sensor 1, the control signal of the CCD constituting a charge-transfer portion relative to the X-ray slit beam imaging plane is differentiated in a normal resolution radiography and other selectable low resolution radiography. More specifically, under the process of a so called bucket-brigade type charge transportation by the charge-transfer portion after executing a normal resolution radiography, the radiography charge of four picture elements may be superimposed at intervals in such a manner that for example four elements arranged in a reticular pattern becomes two picture elements arranged in lengthwise or in crosswise or becomes one picture element.

FIG. 16 shows execution examples of such a binning process. It shows an original image (panoramic image at upper left) obtained as a scout view image, an image (upper right) in which 2×1 binning process is executed for the radiography charge of the same resolution of the original image, an image (lower left) in which 1×2 binning process is executed, and an image (lower right) in which 2×2 binning process is executed. The long image by 2×1 binning process and the wide image by 1×2 binning process can be shown as a correct aspect ratio on the display 15a by a simple image process such as an interleave process. Such an imaging process is generally executed because the obtained image and the image displayed on the display 15a have different resolution and is not become newly required for a binning process.

The resultant reduced exposure dose is achieved as the effect of reducing the X-ray dosage irradiated from the X-ray generator 11 anticipating the increasing amount of the radiography charge which is superimposed after the binning process under the same radiography conditions or by increasing the rotary speed of the rotary means 13a. Although the X-ray exposure dosage is expected to be reduced similarly in either case, a patient being an object to be examined H gets relief from stress in a case wherein the radiography time is shortened by increasing the rotation speed.

Such a binning process can be introduced when a CMOS sensor is adopted as an imaging element. It is briefly explained following a circuit diagram explaining the basic structure of a CMOS sensor.

FIG. 17 is a simplified circuit diagram of 4 picture elements of a CMOS sensor. This circuit includes a capacitor for four picture elements respectively which lie in a reticular pattern between lines LI, LO1, or lines LI2, LO2, MOS transistors M1-M4 constituting switches for reading out the radiography charge stored in each capacitor, sensor amplifiers A1-A3 for generating voltage signals corresponding to the read-out radiography charge, and switches SW1 and SW2 comprised of MOS transistors for selectively connecting with the sensor amplifiers A1-A3.

When a normal radiography is executed with this circuit, the switches SW1 and SW2 are controlled in such a manner that the lines LO1, LO2 are connected with the sensor amplifiers A1 and A2. After obtaining an image, the line K1 is activated, the radiography charges Q1 and Q2 are read out to the lines LO1 and LO2 respectively, the voltage signal produced by the sensor amplifiers A1 and A2 is sampled by an A/D converter (not shown) to convert into digital signals, then the line K2 is activated after the lines LO1 and LO2 once discharge electricity. And the voltage signals corresponding to the radiography charge Q3 and Q4 are generated at the sensor amplifiers A1 and A2 and they are sampled and converted into digital signals. By such operations, the radiography charges Q1-Q4 of all of the picture elements of the CMOS sensor are converted into digital signals.

In case of 2×1 binning process, the switches SW1 and SW2 are controlled in such a manner that the lines LO1, L02 are connected with the sensor amplifiers A1 and A2. After obtaining an image, the lines K1 and K2 are simultaneously activated, the radiography charges Q1 and Q3 are read out to the line LO1 together to be. superimposed and simultaneously the radiography charges Q2 and Q4 are readout to the line L02 together to be superimposed. The sensor amplifier A1 produces the voltage signal corresponding to the superimposed radiography charge Q1+Q3 and the sensor amplifier A2 produces the voltage signal corresponding to the superimposed radiography charge Q2+Q4, so that these voltage signals are sampled and rendered to A/D conversion.

In case of 1×2 binning process, the switches SW1 and SW2 are controlled in such a manner that the lines LO1, LO2 are connected with the sensor amplifier A3. After obtaining an image, the line K1 is activated and the radiography charges Q1 and Q2 are read out to the lines LO1 and LO2 which short-circuit each other to be superimposed. Thus, the sensor amplifier A3 produces the voltage signal corresponding to the superimposed radiography charge Q1+Q2, so that the voltage signal is sampled and rendered to an A/D conversion. Then the line K2 is activated after the lines LO1 and LO2 once discharge electricity and the radiography charges Q3 and Q4 are read out to the lines LO1 and LO2 to be superimposed. As the result the sensor amplifier A3 produces the voltage signal corresponding to the superimposed radiography charge Q3+Q4, so that the voltage signal is sampled and rendered to A/D conversion.

In case of 2×2 binning process, the switches SW1 and SW2 are controlled in such a manner that the lines LO1, LO2 are connected with the sensor amplifier A3. After obtaining an image, the lines K1 and K2 are simultaneously activated, the radiography charges Q1, Q2, Q3 and Q4 are read out to the lines LO1 and LO2 which short-cut each other to be superimposed. The sensor amplifier A3 produces the voltage signals corresponding to the superimposed radiography charges Q1+Q2+Q3+Q4, so that these voltage signals are sampled and rendered to A/D conversion.

Such a binning process for obtaining a scout view image can be introduced into each X-ray imaging apparatus 10 in the above mentioned embodiments. Further, a binning process can be utilized for obtaining a sectional image of an interested area such as a panoramic tomography, a linear tomography and an X-ray CT scan. When the above mentioned binning process is executed for a radiography for obtaining a sectional image, the volume of the image data can be reduced and the data transfer time can be shortened. If such a binning process is appropriately executed considering the performance of the X-ray image sensor 1 itself and the screen size of the display 15a, a convenient X-ray imaging apparatus can be achieved.

The movement of the X-ray generator 11 and the X-ray image sensor 1 (sensor mounting portion 12) respective to the object to be examined H in this invention is a relative movement. Namely, the object to be examined H may be fixed and the X-ray generator 11 and the X-ray image sensor 1 may be moved. Or the X-ray generator 11 and the X-ray image sensor 1 may be fixed and the object to be examined H may be moved.

The movement of the X-ray generator 11 and the X-ray image sensor relative to the object to be examined H in the present invention can be defined by the above mentioned relative movement. For example, if the X-ray generator 11 and the X-ray image sensor 1 are required to be rotated (circulated) relative to the object to be examined H in case of obtaining a sectional image, the object to be examined H may be fixed and the X-ray generator 11 and the X-ray image sensor 1 may be rotated. On the other hand, the X-ray generator 11 and the X-ray image sensor 1 may be fixed and the object to be examined H may be rotated or moved. Further, the rotation or movement of the object to be examined H and the rotation of the X-ray generator 11 and the X-ray image sensor 1 may be combined. The movement other than rotation (circulation) is the same as mentioned.

1. An X-ray image sensor for use in a medical X-ray imaging apparatus comprising a rotary means which has a mounting portion for the X-ray image sensor and holds said X-ray image sensor mounted on said mounting portion and an X-ray generator so as to interpose an object to be examined between said X-ray image sensor and said X-ray generator, wherein
said X-ray image sensor is so constructed as to be mounted on said mounting portion, and comprises an X-ray slit beam imaging plane which is vertically long and has small width, and a CT imaging plane combined with said X-ray slit beam imaging plane, and having larger width than said X-ray slit beam imaging plane, and receiving X-ray cone beam.

2. The X-ray image sensor as set forth in claim 1, wherein said CT imaging plane is combined with said X-ray slit beam imaging plane in a manner that said CT imaging plane intersects with said X-ray slit beam imaging plane.

3. The X-ray imaging sensor as set forth in claim 1 or 2, wherein said X-ray slit beam imaging plane has a longitudinal dimension required for a cephalometric radiography, and is so constructed as to be masked in a part of said X-ray slit beam imaging plane with a shielding member when a panoramic radiography or a liner tomography is executed.

4. The X-ray imaging sensor as set forth in claim 1 or 2, both of said CT imaging plane and said X-ray slit beam imaging plane are composed of any one selected from a MOS sensor, a CMOS sensor, a TFT sensor, an X-ray solid-state image sensing device, and a CCD sensor.

5. An X-ray imaging apparatus, comprising a rotary means holding an X-ray generator for interposing an object to be examined together with said imaging sensor as set forth in claim 1 or 2, and an orbit control means for moving said rotary means along any one of different kinds of orbits prepared in advance in order to produce an X-ray image of said object for different diagnosis purposes.

6. The X-ray imaging apparatus as set forth in claim 5,
wherein said orbit control means moves said rotary means relative to said object along the orbit respectively for performing at least two types of tomography selected from a linear tomography, a panoramic tomography, a cephalometric radiography, and an X-ray CT scan.

7. The X-ray imaging apparatus as set forth in claim 5, wherein said X-ray imaging apparatus has an imaging means for producing a scout view image, and wherein the resolution is reduced in case of producing a scout view image.

8. The X-ray imaging apparatus as set forth in claim 5, wherein said X-ray imaging apparatus has an imaging means for executing any one of a linear tomography, a panoramic tomography, a cephalometric radiography, and an X-ray CT scan, and wherein the resolution is reduced in case of producing an image by way of any one of said linear tomography, said panoramic tomography, said cephalometric radiography, and said X-ray CT scan.

9. The X-ray imaging sensor as set forth in claim 3, both of said CT imaging plane and said X-ray slit beam imaging plane are composed of any one selected from a MOS sensor, a CMOS sensor, a TFT sensor, an X-ray solid-state image sensing device, and a CCD sensor.
